(19) United States
(12) Patent Application Publication
(10) Pub. No.: US 2009/0294732 A1
(43) Pub. Date: Dec. 3, 2009

(54) COATINGS FOR MEDICAL DEVICES HAVING REVERSIBLE HYDROPHOBIC TO HYDROPHILIC PROPERTIES

(75) Inventors: Liliana Atanasoska, Edina, MN (US); Michel Arney, Minneapolis, MN (US); Robert Warner, Woodbury, MN (US); Shankar Godavarti, St. Paul, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: BOSTON SCIENTIFIC SCIMED, INC., Maple Grove, MN (US)

(21) Appl. No.: 12/129,038

(22) Filed: May 29, 2008

(51) Int. Cl. H01B 1/20; A61M 25/01; A61M 25/10; A61M 25/09

(52) U.S. Cl. 252/500; 604/265; 604/103.07; 604/96.01; 600/585

(57) ABSTRACT

A medical device including a coating disposed on at least a portion of a surface of the medical device, the coating including at least one polymer material having reversible wettability.

### FIELD OF THE INVENTION

The present invention relates to coatings for use on medical devices, in particular, those coatings which provide the medical device with lubricity, to devices having such coatings disposed thereon, and to methods of making and using the same.

### BACKGROUND OF THE INVENTION

Lubricious coatings are commonly employed on insertable or implantable medical devices to reduce the coefficient of friction which consequently reduces the discomfort caused to a patient during a procedure.

Lubricious coatings can include both hydrophobic and hydrophilic polymers. Hydrophilic polymers have become more desirable because they tend to be more biocompatible or blood compatible, are more readily discharged from the body and have less of a tendency to cause tissue irritation than hydrophobic polymers.

One class of polymers popularly used in lubricious coatings dissolve or swell in an aqueous environment, and are capable of manifesting lubricity while in a wet state. These polymers are often referred to in the art as “hydrogels”. When hydrated, these substances have low frictional forces in humoral fluids including saliva, digestive fluids and blood, as well as in saline solution and water. Such substances include polyethylene oxides (optionally linked to the substrate surface by interpenetrating network, IPN, with poly(meth)acrylate polymers or copolymers; copolymers of maleic anhydride; (meth)acrylamide polymers and copolymers; (meth)acrylic acid copolymers; poly(vinyl pyrrolidone) and blends or interpolymers with polyurethanes; and polysaccharides.

These water soluble coating materials, while popular because they provide excellent lubricity and biocompatibility, may be sensitive to moisture and can prematurely absorb ambient moisture which causes surface stickiness or tackiness. This can produce a “self adhesion” effect, which is an undesirable adhesion of the medical device to itself via the coating, to other devices, or to any other surface to which it comes in contact during sterilization or storage. In the case of dilatation balloons, after sterilization or storage hydrogel coatings on the folded section of the balloon can stick to themselves. This will lead to pinhole failure upon expansion of the balloon.

### SUMMARY OF THE INVENTION

In one aspect, the present invention relates to a lubricious coating for a medical device including a polymeric material having reversibly switchable wettability between hydrophilic and hydrophobic states.

In some embodiments, the coating includes a conductive polymer.

In some embodiments, the conductive polymer is doped with a low surface energy dopant.

In another aspect, the present invention relates to a method of providing at least a portion of a surface of a catheter assembly with reversibly switchable wettability, the method including providing a dynamic polymer layer, providing an electrolyte that is in contact with the dynamic polymer layer, providing a conductive substrate layer electrically connected to a power source and electrically coupled to the dynamic polymer layer. Electrolyte fluxes in and out of the dynamic polymer layer upon application of electrical potential across the electrolyte between the conductive substrate layer and the dynamic polymer layer, resulting in changes in the dynamic polymer layer between hydrophilic and hydrophobic states.

The coatings may be employed on any insertable or implantable medical device including, but not limited to, any catheter assembly or component thereof, in particular, to expandable medical balloons, catheter shafts, distal tips.

These and other aspects, embodiments and advantages of the present invention will become immediately apparent to those of ordinary skill in the art upon review of the Detailed Description and Claims to follow.

### BRIEF DESCRIPTION OF THE DRAWINGS

FIG. 1 is a schematic cross-sectional diagram illustrating a simple actuation device for reversibly switching the wettability of a polymer layer;

FIG. 2 is a schematic diagram illustrating an embodiment of an electroactive polymer actuator in use;

FIG. 3 is a schematic diagram illustrating an embodiment of an actuator for reversibly switching the wettability of a polymer layer which may be employed in accordance with the present invention;

FIG. 3a is a schematic diagram illustrating an embodiment of an actuator for reversibly switching the wettability of a polymer layer which may be employed in accordance with the present invention.

FIG. 4 is a longitudinal cross-section of a catheter assembly;

FIG. 5 is a longitudinal cross-section of a balloon having a lubricious coating including a polymer having switchable wettability;

FIG. 5A is a radial cross-section taken at section 5A-5A in FIG. 5.

FIG. 6 is a partial longitudinal cross-section of an expandable medical balloon having distal tip having a lubricious coating including a polymer having switchable wettability.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

All US patents and applications and all other published documents mentioned anywhere in this application are incorporated herein by reference in their entirety. Any copending patent applications, mentioned anywhere in this application are also hereby expressly incorporated herein by reference in their entirety.

The present invention relates to a lubricious coating for a medical device, the lubricious coating including a polymer having switchable wettability.

In some embodiments, the polymers useful herein are electrochemically active polymers that are susceptible to electrochemistry that either alters a redox state of the polymer, i.e. is either oxidized or reduced, or where there is a redistribution of charges within the polymer so that one end is reduced and the other end is oxidized.

The present invention relates to a lubricious coating for a medical device, the lubricious coating including a polymer having switchable wettability. Suitable polymer materials include electronic/conducting polymers which are typically conjugated, and are also sometimes referred to as “synthetic metals”. Examples include, but are not limited to, poly(acetylene)s, poly(pyrrole)s, poly(N-methyl pyrrole), poly(thiophene)s, poly(alkyl thiophenes), poy(aniline)s, poly(fluorene)s, poly(3-hexylthiophene), polynaphthalenes, poly(p-phenylene sulfide), poly(heteroaromatic vinylenes), poly(para-phenylene vinylene)s, poly(furan)s, polyazulenes, polyquinones, polystyrene sulfonate, polyethylenedioxythiophenes, poly(p-phenylenes), poly(p-phenylene vinylene)s, polysulfones, poly(pyridine)s, poly(quinoxaline)s, polyanthraquinones, poly(n-vinylcarbazole)s, poly(azulene)s, poly(acene)s, etc., to mention only a few, as well as suitable derivatives thereof

The poly(heteroaromatic vinylenes) may have the following formula:

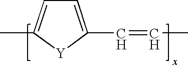

where Y═NH, NR, S or O. See also U.S. Pat. No. 4,900,782, for poly(heteroaromatic vinylene) polyelectrolytes, the entire content of which is incorporated by reference herein.

Typically, such conductive polymers include a positively charged conjugated backbone, and negatively charged counterions referred to as dopants. The selection of dopants can determine the degree of wettability that may be achieved.

Examples of dopants which may be employed herein include, but are not limited to, perfluorooctanesulfonate (PFOS), alone or in combination with perchlorate (ClO₄—), for example. For example, poly(pyrrole), in the oxidized state with PFOS, as the dopant, is highly hydrophobic, and in the neutral state, highly hydrophilic. In some embodiments, it may be suitable to employ mixtures of dopants for obtaining intermediate properties.

Suitably, the polymer materials employed herein exhibit what is referred to in the art as “superhydrophobicity” and “superhydrophilicity”. As used herein, the term “superhydrophobicity”, shall be used to refer to polymer materials, which when applied to the surface of a medical device as described herein, exhibit a water contact angle of about 145° or greater, suitably about 150° or greater, suitably up to about 180°. In some embodiments, the coating in the “superhydrophobic” state may exhibit a contact angle of between about 150° and about 160°.

The term “superhydrophilicity”, as employed herein, shall refer to those polymer materials that produce a surface exhibiting water contact angles of about 10° or less, suitably about 5° or less. In some embodiments, the polymer materials employed can produce a surface having a water contact angle approaching 0°.

Suitably, the surface may be reversibly switched between a superhydrophobic state and a superhydrophilic state using appropriate external stimuli such as, for example, electrical potential, temperature, light illumination, adsorption of biopolymer, and treatment with selected solvents. For polymer materials that have temperature sensitive hydrophobicity/hydrophilicity see U.S. Patent Publication No. 2006/0115623, the entire content of which is incorporated by reference herein.

Stimuli via electrical potential has been found to be particularly desirable because it is simple, readily controlled by electricity, and the switch from superhydrophobic to superhydrophilic can be rapidly induced. Some forms of external stimuli may take days to induce the switch, a scenario which may not be as desirable wherein the application is an insertable and/or implantable medical device.

In order to bring about reversibly switchable wettability in a polymer layer, the following components are utilized: (a) a power source (e.g. a battery), (b) a dynamic polymer layer including a polymer having the capability of exhibiting reversibly switchable wettability upon application of external stimuli; (c) a counter electrode and (d) an electrolyte in contact with both the dynamic polymer and the counter electrode. This will be illustrated in more detail by the following figures.

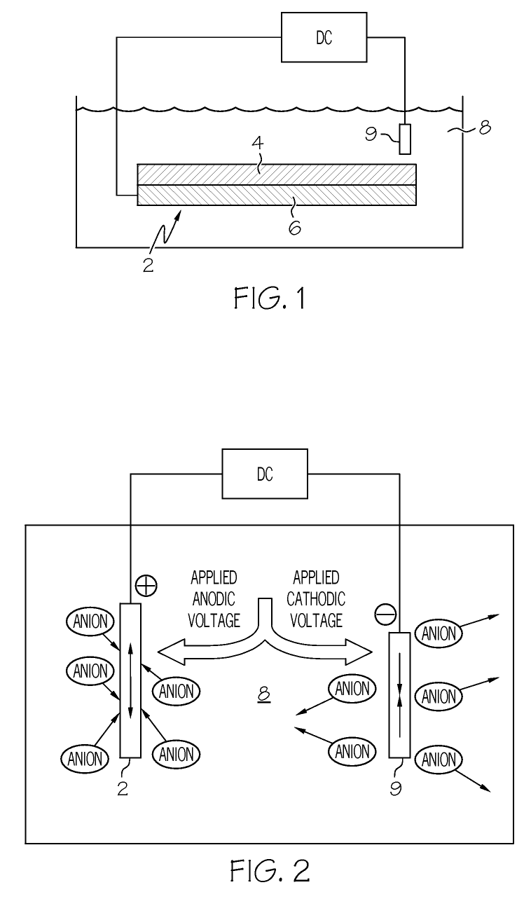

Referring now the drawings, FIG. 1 is a schematic cross-sectional diagram of an actuation device 2 for purposes of illustrating each of the elements commonly utilized in inducing reversibly switchable wettability. As an example of such a device, or modifications thereof, refer to WO/2005/053836, the entire content of which is incorporated by reference herein.

Actuation device 2 includes a dynamic polymer layer 4 having reversibly switchable wettability. Dynamic polymer layer 4 is coupled to a conductive substrate layer 6 which is shown in FIG. 1 as continuous with dynamic polymer layer 4. However, conductive substrate layer 6 need not be continuous with polymer layer 4, and could be in the form of a mesh, as well, for example. Actuator 2 includes a counter electrode 9. In this embodiment, polymer layer 4 is shown immersed in an electrolyte solution 8 for purposes of discussing the features of an actuation device 2 only for reversibly switchable wettability. Electrolyte 8 may be, for example, a liquid, a gel, or a solid, so long as ion movement is allowed, some embodiments of which will be discussed in more detail below. One example of a liquid electrolyte is a saline-based contrast solution.

It should be noted that typically, it is not desirable for the conductive substrate layer 6 to be in direct contact with an electrolyte 8 because it may corrode or react in the presence of an electrolyte.

Placing polymer layer 4 in contact with electrolyte 8, allows free ions can diffuse into or out of polymer layer 4.

Electrolyte 8 may come into contact with only a portion of the surface of polymer layer 4, or up to the entirety of the surface of polymer layer 4 as shown in FIG. 1. Suitably, the conductive substrate layer 6 is not placed in direct contact with electrolyte 8. Furthermore, electrolyte 8 can be encapsulated within the polymer layer 4.

In this embodiment, polymer layer 4 is shown in film form. The conductive polymer could be provided in other forms such as fibers or groups of coiled or bundled fibers or a combination of film and fibers, etc.

Any number of procedures may be employed to provide polymer layer 4 with a conductive substrate layer 6 including, but not limited to, sputtering, gilding, casting, etc. the polymer onto a metal substrate, electrochemically depositing the polymer onto the metal, thermal evaporation, vapor deposition, etc. For further discussion of this technique, see U.S. Pat. No. 6,982,514 and U.S. Publication No. 2005/0178286, each of which is incorporated by reference herein. See also Xu, Lianbin et al., Reversible conversion of Conducting Polymer Films from Superhydrophobic to Superhydrophilic, Angew. Chem. Int. Ed., Vol. 44, pages 6009-6012 (2005), the entire content of which is incorporated by reference herein wherein a one-step fabrication process using a combination of electrochemical and chemical polymerization techniques to galvanostatically apply a film of conductive polymer to a metallic or metalized substrate. For example, polypyrrole or polyaniline can be galvanostatically polymerized with low-surface energy dopant, perfluorooctane sulfonate onto a metalized substrate such as a platinized substrate or gold coated substrate for example. See also D. Zhou et al., “Actuators for the Cochlear Implant,” Synthetic Metals 135-136 (2003) 39-40, the content of which is incorporated by reference herein, for galvanostatic polymerization of polypyrrole onto a platinized substrate.

Conductive substrate layer 6 may act as the working electrode. Conductive substrate layer 6 may be formed from any suitable conductive material such as another conducting polymer or a metal such as gold (Au) or platinum (Pt), or a metal alloy, for example.

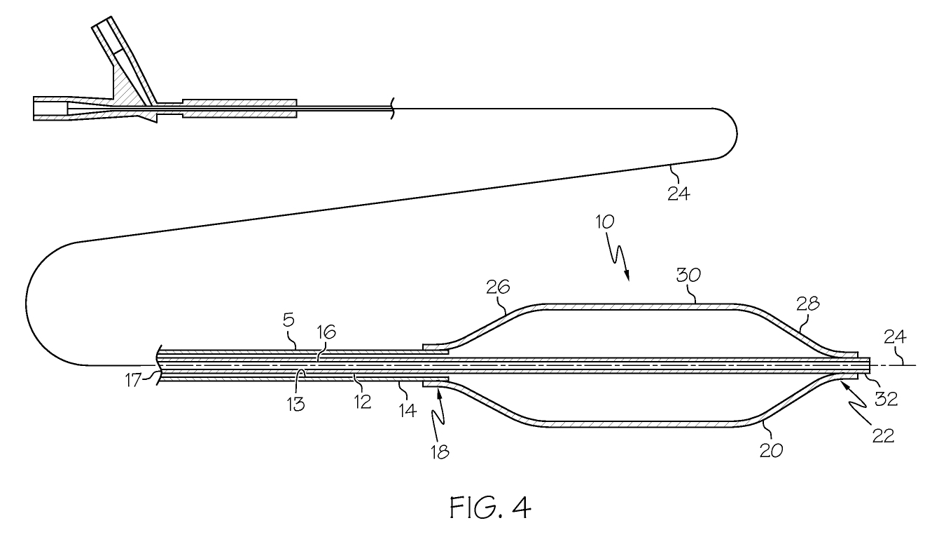

Conductive substrate layer 6 is in electrical connection with a voltage supply 11. A counter electrode 9, submersed other otherwise in contact with electrolyte 8, is also shown in contact with a voltage supply 11, and completes the electrical circuit. See FIGS. 4a and 4b, for example, of U.S. Pat. No. 6,982,514.

Counter electrode 9 is in electrical contact with electrolyte 8 in order to provide a return path for charge to a source 11 of potential difference between polymer layer 4 and electrolyte 8. The counter electrode 9 may be formed using any suitable electrical conductor, for example, another conducting polymer, a conducting polymer gel, or a metal, such as stainless steel, gold, platinum, copper, etc. At least a portion of the surface of the counter electrode 9 is in contact with the electrolyte 8, in order to provide a return path for charge.

Counter electrode 9 may be in the form of a wire, such as a wire winding, or may be applied by any suitable means including electroplating, chemical deposition, or printing. In order to induce switching of the polymer layer 4 between its oxidation and reduction states, a current is passed between polymer layer 4 and counter electrode 9, inducing oxidation or reduction of the electronic polymer in the polymer layer 4.

The source of electrical potential for use in connection with the present invention can be quite simple, consisting, for example, of a dc battery or other direct current source and an on/off switch. Alternatively, more complex systems can be utilized. For example, an electrical link can be established with a microprocessor, allowing a complex set of control signals to be sent to the polymer layer 4.

Electrolytes can be induced to flow into and out of polymer layer 4 resulting in oxidation and reduction, i.e. redox reactions, occurring in polymer layer 4 in response to external stimuli, in this embodiment, changes in electrical potential. This in turn results in changes in the water contact angle of polymer layer 4, i.e. surface energy changes and surface tension changes. Polymer layer 4 can also expand or contract upon influx or outflow of electrolyte into the layer. FIG. 2 is a schematic diagram illustrating such an actuator in use.

In this embodiment, polymer layer 4 includes at least one electronic polymer. Examples of such polymers are provided above and include, among others, poly(pyrrole)s, poly(aniline)s, and poly(thiophene)s, with poly(pyrrole)s being a particularly suitable polymer due to biocompatibility. Other suitable polymers were previously discussed above. Polymer layer 4 can include only the conductive polymer, or a mixture of conductive polymers, or a mixture of conductive polymer and non-conductive polymer.

As a specific example, poly(pyrrole) (PPy) is included in layer 4. In one specific embodiment, the polymer layer 4 includes a poly(pyrrole) polymer (PPy), and an electrolyte having low surface energy anions such as perfluorooctane sulfonate (PFOS) or a combination of PFOS and ClO₄—. The polymer layer 4 can be doped and dedoped (oxidized and reduced) by changing the electrical potential. An example of this reaction is provided below:

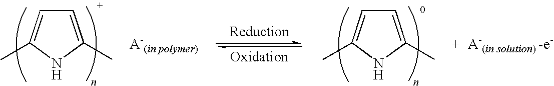

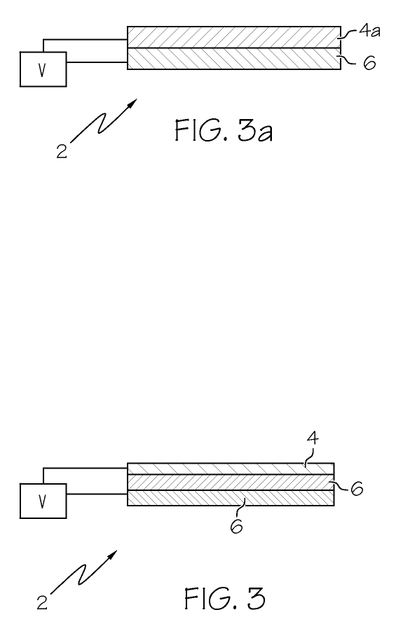

Examples of alternative configurations of actuators in which a solid or gel polyelectrolyte are employed, or the polymer layer 4 has been predoped with electrolyte are shown in FIGS. 3 and 3a.

FIG. 3 illustrates an alternative embodiment wherein a gel or solid electrolyte layer 8 is provided between conductive substrate layer 6 and polymer layer 4. It is also possible to provide electrolyte 8 as a separate component applied at will when it is desirable to induce the polymer layer 4 to undergo a switch. The electrolyte need only be in contact with at least a portion of the surface of the dynamic polymer layer 4 to allow for the flow of ions and thus acts as a source/sink for the ions. Any suitable electrolyte may be employed herein. However, for purposes of providing hydrophobicity to the electronic polymers, low-surface energy dopants find most utility.

In another embodiment, actuator 2 can be formed from an electronic polymer which has been previously doped with anion such as a polypyrrole doped with PFOS or a combination of PFOS and perchlorate (ClO₄—). See Xu et al., Reversible Conversion of Conducting Polymer Films from Superhydrophobic to Superhydrophilic.

FIG. 3a illustrates an embodiment of an actuator 2 wherein polymer layer 4a includes a predoped polymer such as poly(pyrrole) predoped with PFOS. Polymer layer 4a is disposed on a conductive layer 6. The predoped polymer is in an oxidized hydrophobic state. Prior to or during use, the polymer layer 4 can be dedoped by applying a negative potential, such as about −0.6 V vs. the counter electrode 9, for example, an Ag/AgCl counter electrode, for a short period of time, causing the anions to flow from the polymer layer 4. This induces the neutral state of poly(pyrrole) which is slightly hydrophilic.

These polymers are typically stimulated by low voltages of less than about 2V. For example, PPy is preferably biased from about −0.8V to about +0.6V while poly(3-methyl thiophene) may suitably be biased from about +0.2V to about +0.8V, poly(paraphenylene) from about −2V to about +1.1V and polyaniline from about +0.1V to about +0.7V. Derivatized polythiophenes may be biased from about +1.5V to about −1.5V depending on the substitutions made. See, for example, US 2005/0178286.

Superhydrophobicity and superhydrophilicity of these electrolytic polymers, such as poly(pyrrole), can be achieved by providing the surface with surface characteristics such as pores, canals or the like. For example, in Xu et al., Reversible Conversion of Conducting Polymer Films from Superhydrophobic to Superhydrophilic, poly(pyrrole) was polymerized onto a conducting surface using a combination of electrochemical and chemical polymerization processes. Chemical polymerization can be induced by the addition of Lewis Acids such as FeCl₃ or ammonium persulfate along with codopants such as NaClO₄. See U.S. Patent Publication No. 2005/0178286, and see L. Xu et al., Reversible Conversion of Conducting Polymer Films from Superhydrophobic to Superhydrophilic, Angew. Chem. Int. Ed., Vol. 44, pages 6009-6012 (2005).

The presence of Lewis Acids results in a highly porous PPy coating. It has been found that providing a film with high porosity can enhance both the hydrophobicity and hydrophilicity of the surface to which the film is applied. In fact, it is beneficial to have a combination of coarse and fine surface roughness, i.e. pore sizes of a smaller size range of about 1 microns to about 5 microns, as well as larger pore sizes of about 10 to about 50 microns. A small amount of Fe in the plating solution has been found to play a key role in the formation of the highly porous structure. Examples of Fe salts that may be employed include, but are not limited to, FeCl₃, FeCl₂, Fe(ClO₄)₃, Fe(ClO₄)₂, FeSO₄, etc.

While some actuator configurations have been discussed above, many different configurations are available for forming such actuation devices. See, for example, WO/2005/053836. Other configurations can be found in U.S. Pat. No. 6,249,076, the entire content of which is incorporated by reference herein.

Thus, in this fashion, the surface wettability of a medical device can be controlled, i.e. switched, by changing the electrical potential wherein the polymer switches between the doped (oxidized) state and the dedoped (neutral) state.

The present invention can be employed on any type of medical device to provide lubricity (wet) to the surface of the device during use and to provide a hydrophobic surface during storage and handling. This can prevent the coating from becoming prematurely wet which can lead to self-adhesion and blocking, and consequently, damage to the coating.

The invention may find utility for use on any catheter device or component thereof Examples of medical device surface regions benefiting from the present invention include, but are not limited to, outside and/or luminal surfaces of insertable and/or implantable medical devices such as vascular devices such as vascular catheters including, for example, balloon catheters and any components thereof, urinary catheters, ureteral stents, hydrolyser catheters, guide wires, pullback sheaths, filters including vena cava filters, left ventricular assist devices, endoscopic devices, endotracheal devices including airway tubes, drug delivery devices, injection needles, artificial hearts, drainage tubing, gastroenteric tubing, colonoscopic tubing, neural catheters and wires, etc.

FIG. 4 illustrates generally at 10, a catheter assembly, various portions of which may include a coating as described above. Catheter assembly 10 includes a shaft assembly 5 including inner shaft 12 and outer shaft 14 and an inflation lumen 13 therebetween. Proximal waist 16 of balloon 20 is disposed about distal end of outer shaft 14 and distal waist 18 of balloon 20 is disposed about distal end of inner shaft 12. Inner surface 16 of inner shaft 12 defines a guide wire lumen 17 through which guide wire 22 is disposed.

Any portion of catheter assembly 10 may include a dynamic polymer region 100 according to the invention including guide wire 24, outer shaft 14, balloon waist portions 18, 20, cone portions 26, 28 and body portion 30, as well as the exposed distal portion 32 of inner shaft 14, or any combination thereof The dynamic polymer region 100 referred to herein, shall be intended to include all of the components necessary to provide the region with reversibly switchable wettability including the dynamic polymer layer 4, the conductive substrate layer 6 and electrolyte 8.

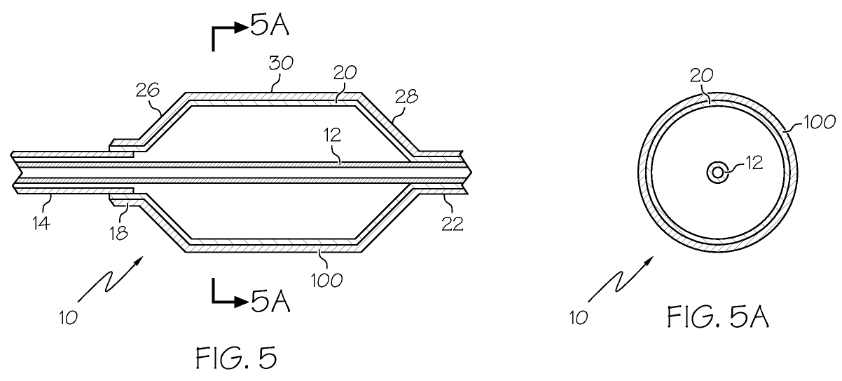

FIG. 5 is an enlarged longitudinal cross-section of an expandable balloon 20 similar to that shown in FIG. 4. Balloon 20 is shown having a dynamic polymer region 100 according to the invention disposed on waist portions 18, 22, cone portions 26, 28 and body portion 30. However, any portion of balloon 20 can be selectively provided with such an actuator as desired.

For example, in one embodiment, it may be desirable to dispose dynamic polymer region 100 on waist portions 18, 22 and cone portions 26, 28, but not on body portion 30.

FIG. 5a is a radial cross-section taken at section 5A-5A in FIG. 5.

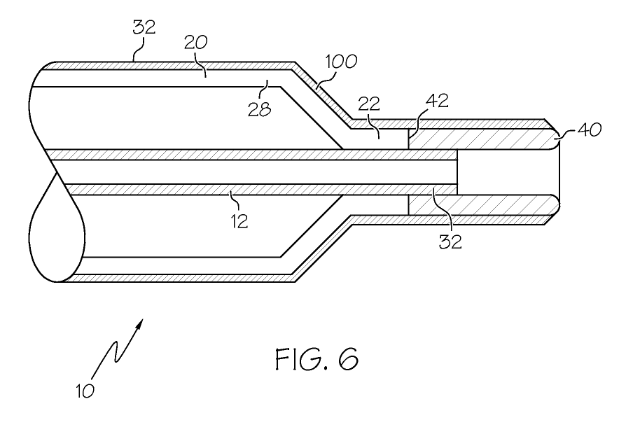

FIG. 6 is a partial longitudinal cross-section showing only the distal portion of balloon 20 disposed about inner shaft 12 of catheter assembly 10. The catheter assembly in this embodiment, is further equipped with a distal tip 40 disposed about the most distal portion 32 of inner shaft 12 and abutting balloon waist 22 at 42.

In this embodiment, dynamic polymer region 100 is disposed about balloon body 30, cone 28 and waist portion 22, as well as about distal tip 40. Dynamic polymer region 100 may be disposed about the portions of balloon 20 that are not shown in FIG. 5, such as waist 18 and cone 26 portions shown in FIG. 5. Of course, dynamic polymer region 100 may disposed about any portion or portions as discussed above. For example, it may be desirable to disposed dynamic polymer region on distal tip 40 only, or on distal tip, waist 22 and cone 28, or other combinations as well.

The invention is not limited by where on the catheter assembly or other medical device the coating is disposed. Furthermore, it may be desirable to provide a coating having a gradient of lubricity, for example, more lubricious on the distal cone 22 and waist 28, and getting exceedingly less lubricious on the body 30 to the proximal waist 18 and cone 26 portions.

The dynamic polymer region can be deposited on the desired medical device surface using any suitable method known in the art including, but not limited to, extrusion, casting, dip coating, spin coating, or electro-polymerization/deposition techniques. Such dynamic polymer layers can also be patterned, for example, using lithographic techniques, if desired. See also US 2005/0178286 and Xu et al., Reversible Conversion of Conducting Polymer Films from Superhydrophobic to Superhydrophilic.

The above disclosure is intended to be illustrative and not exhaustive. This description will suggest many variations and alternatives to one of ordinary skill in this art. Those familiar with the art may recognize other equivalents to the specific embodiments described herein which equivalents are also intended to be encompassed by the claims.

1. A medical device comprising a coating disposed on at least a portion of a surface of said medical device, the coating comprising at least one polymer material having reversibly switchable wettability.

2. The medical device of claim 1 wherein said at least one polymer is an electronic polymer that exhibits reversibly switchable wettability upon application of different electrical potential.

3. The medical device of claim 1 wherein the electrical potential ranges from about −0.8V to about +0.8V.

4. The medical device of claim 1 wherein said at least said portion of said surface comprising said coating has a water contact angle from 0° to about 160°.

5. The medical device of claim 1 wherein said surface comprising said coating has a water contact angle that is switchable from less than about 50° to greater than about 145°.

6. The medical device of claim 1 wherein said surface comprising said coating has a water contact angle that is switchable from less than about 5° to greater than about 150°.

7. The medical device of claim 1 wherein said surface comprising said coating has a water contact angle that is switchable between about 0°-5° and about 150-180°.

8. The medical device of claim 1 wherein said at least one polymer material is a member selected from the group consisting of poly(acetylene)s, poly(pyrrole)s, poly(N-methyl pyrrole), poly(thiophene)s, poy(aniline)s, poly(fluorene)s, poly(3-hexylthiophene), polynaphthalenes, poly(p-phenylene sulfide), poly(para-phenylene vinylene)s, poly(furan)s, derivatives thereof and mixtures thereof.

9. The medical device of claim 1, said coating comprising poly(pyrrole).

10. The medical device of claim 9 wherein said poly(pyrrole) is doped with perfluorooctane sulfonate.

11. The medical device of claim 1 wherein said coating comprises a porous structure.

12. The medical device of claim 11 wherein said porous structure comprises coarse and fine roughness.

13. The medical device of claim 12 wherein said porous structure comprises pore sizes ranging from about 1 to about 5 microns and from about 10 to about 50 microns.

14. The medical device of claim 1 wherein said medical device is a catheter assembly.

15. The medical device of claim 14 wherein said catheter assembly comprises at least one catheter shaft having a proximal end and a distal end and a distal tip engaged with said distal end of said catheter shaft, said coating disposed on at least a portion of a surface of said expandable member.

16. The medical device of claim 1 wherein said medical device is an expandable member, said coating disposed on at least a portion of a surface of said expandable member.

17. The medical device of claim 1 wherein said medical device is an expandable balloon, said expandable balloon comprising a body, waist and cone portions, said coating disposed on at least the waist portions.

18. The medical device of claim 18 wherein said coating is disposed on at least said waist and cone portions.

19. The medical device of claim 1 wherein said medical device is a guide wire.

20. A method of providing at least a portion of a surface of a catheter assembly with reversibly switchable wettability, the method comprising:
providing a dynamic polymer layer;
providing an electrolyte, said electrolyte in contact with said dynamic polymer layer;
providing a conductive substrate layer electrically connected to a power source, said conductive substrate layer electrically coupled to said dynamic polymer layer;
wherein said electrolyte fluxes in and out of said dynamic polymer layer upon application of electrical potential across the electrolyte between the conductive substrate layer and the dynamic polymer layer, and said dynamic polymer layer exhibits hydrophilic and hydrophobic states.
